(19) United States
(12) Patent Application Publication
(10) Pub. No.: US 2008/0177322 A1
(43) Pub. Date: Jul. 24, 2008

(54) SPINAL STABILIZATION SYSTEMS AND METHODS

(76) Inventors: Melissa Davis, Austin, TX (US); K. Scott Ely, Cedar Park, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/959,063

(22) Filed: Dec. 18, 2007

Related U.S. Application Data
(60) Provisional application No. 60/882,818, filed on Dec. 29, 2006.

(51) Int. Cl. A61B 17/58; A61B 17/56; A61B 17/08

(52) U.S. Cl. 606/266; 606/275

(57) ABSTRACT

A spinal stabilization system includes bone fastener assemblies to be coupled to vertebrae. Each bone fastener assembly includes a bone fastener and a collar. The bone fastener has a head portion having at least a first cross-sectional shape in a first plane, and a second cross-sectional shape in a second plane. The collar has a circular opening in the bottom, with a relief extending from the circular opening. The second cross-sectional shape of the bone fastener is keyed to the opening to permit insertion of the bone fastener into the collar assembly from the bottom. After insertion, the bone fastener is rotated to prohibit removal of the bone fastener from the collar. The collar can then be rotated and/or angulated relative to the bone fastener. An elongated member can be positioned in the collar and a closure member is then used to secure the elongated member to the collar.

### CROSS-REFERENCE TO RELATED APPLICATIONS

This application claims the benefit of U.S. Provisional Application No. 60/882,818, filed Dec. 29, 2006.

### BACKGROUND OF THE INVENTION

1. Field of the Invention

The present invention generally relates to a polyaxial fastener assembly, and spinal stabilization systems that include at least one polyaxial fastener. Embodiments of the invention relate to spinal stabilization systems that can be inserted into a patient preferrably using a minimally invasive surgical procedure. Embodiments of the invention relate to methods of assembling implant system components, methods of assembling stabilization systems and components, as well as the methods and tools employed for performing minimally invasive spinal stabilization procedures.

2. Description of Related Art

Bone can be subject to degeneration caused by trauma, disease, and/or aging. Degeneration can destabilize bone and affect surrounding structures. For example, destabilization of a spine can result in alteration of a natural spacing between adjacent vertebrae. Alteration of a natural spacing between adjacent vertebrae can subject nerves that pass between vertebral bodies to pressure. Pressure applied to the nerves can cause pain and/or nerve damage. Maintaining the natural spacing between vertebrae can reduce pressure applied to nerves that pass between vertebral bodies. A spinal stabilization procedure can be used to maintain the natural spacing between vertebrae and promote spinal stability.

Spinal stabilization can involve accessing a portion of the spine through soft tissue. Conventional stabilization systems can require a large incision and/or multiple incisions in the soft tissue to provide access to a portion of the spine to be stabilized. Conventional procedures can result in trauma to the soft tissue, for example, due to muscle stripping.

Spinal stabilization systems for a lumbar region of the spine can be inserted during a spinal stabilization procedure using a posterior spinal approach. Conventional systems and methods for posterolateral spinal fusion can involve dissecting and retracting soft tissue proximate the surgical site. Dissection and retraction of soft tissue can cause trauma to the soft tissue, and extend recovery time. Minimally invasive procedures and systems can reduce recovery time as well as trauma to the soft tissue surrounding a stabilization site.

U.S. Pat. No. 6,530,929 to Justis et al. (hereinafter “Justis”), which is incorporated by reference as if fully disclosed herein, describes minimally invasive techniques and instruments for stabilizing a bony structure in an animal subject. Justis provides a method for using an instrument to connect at least two bone anchors with a connecting element. The instrument is secured to the anchors and manipulated to place the connecting element in a position more proximate the anchors.

U.S. Patent Application Publication No. 20060084993, which is incorporated by reference as if fully disclosed herein, describes a spinal stabilization system including bone fastener assemblies having a bone fastener and a collar. The collar can be rotated and/or angulated relative to the bone fastener. Detachable members can be coupled to the collar to allow for formation of the spinal stabilization system through a small skin incision. The detachable members can allow for alignment of the collars to facilitate insertion of an elongated member in the collars.

### SUMMARY OF THE INVENTION

The purpose and advantages of the present invention will be set forth in and apparent from the description that follows, as well as will be learned by practice of the invention. Additional advantages of the invention will be realized and attained by the methods and systems particularly pointed out in the written description and claims hereof, as well as from the appended drawings.

To achieve these and other advantages and in accordance with the purpose of the invention, as embodied and broadly described, the invention includes a spinal stabilization system to be installed in a patient to stabilize a portion of a spine. The spinal stabilization system can be installed using a minimally invasive procedure. An instrumentation kit can provide instruments and spinal stabilization system components necessary for forming a spinal stabilization system in a patient.

The invention also includes a spinal stabilization system that can be used to achieve rigid pedicle fixation while minimizing the amount of damage to surrounding tissue. In some embodiments, a spinal stabilization system can be used to provide stability to two or more vertebrae. A spinal stabilization system can include an elongated member, two or more bone fastener assemblies, and/or a closure member. The bone fastener assembly can include, but is not limited to, a bone fastener and a collar (or tulip). A first portion of the bone fastener can couple to a portion of the spine during use. A first portion of a collar (or tulip) includes a socket which can couple to a second portion of the bone fastener. A second portion of the collar can couple to an elongated member during use. In some embodiments, an orientation of the bone fastener can be independent of the orientation of the collar for a bone fastener assembly. After the bone fastener is placed in a vertebral body, the collar coupled to the bone fastener can be positioned so that the elongated member can be positioned in the collar and in at least one other collar that is coupled to another vertebral body by a bone fastener.

In accordance with one aspect of the invention, a bone fastener assembly includes a bone fastener, and a collar. The collar has an upper portion with a slot to receive an elongated member, a lower portion having a socket formed therein and an opening with a predetermined shape in communication with the socket, and a first longitudinal axis extending through the upper and lower portions. The bone fastener has a head portion to be received in the socket, and a shank portion to be attached to a vertebrae. The head portion has a first cross-sectional shape in a plane generally perpendicular to a second longitudinal axis extending through the head and shank portions of the fastener. The head portion further includes a second cross-sectional shape in a plane angled relative to the second longitudinal axis. The first cross-sectional shape is configured to prohibit movement of the head portion through the opening in the collar and the second cross-sectional shape is configured to allow movement of the head portion through the opening.

The head is positioned in a socket of the collar through the opening in bottom surface of the collar under a particular orientation with respect to the collar. In a preferred embodiment, the predetermined shape of the opening includes a first portion having a generally circular shape in a plane perpendicular to the first longitudinal axis, and a second portion having a relief extending from the generally circular shape. In this embodiment, and in order to permit insertion of the fastener into the collar, the fastener is oriented such that the second cross-sectional shape of the fastener is keyed to register with the circular shape and relief of the opening.

Further, once the fastener is received within the socket of the collar, separation of the fastener from the collar is inhibited by rotating the fastener to a different orientation with respect to the collar. Indeed, the fastener can be rotated substantially to any angle to reposition the first cross-sectional shape of the head portion out of alignment with the relief. The fastener therefore can be angulated, about a plurality of axes, within the collar (i.e., the bone fastener can move polyaxially relative to the collar within a defined range of motion) without risk of removal from the collar or the socket therein.

In an embodiment, a collar includes, but is not limited to, arms and a body to form a slot to receive an elongated member. When the elongated member is positioned in the collar, a portion of the elongated member can engage or otherwise be coupled to a head of a bone fastener of the bone fastener assembly to lock the position of the various components.

Inner surfaces of the arms of a bone fastener assembly collar can include a thread to engage a complementary thread of a closure member. A closure member secures the elongated member to the bone fastener assembly, and secures the position of the various components. In a preferred embodiment, a modified thread configuration is used.

It is to be understood that both the foregoing general description and the following detailed description are exemplary and are intended to provide further explanation of the invention claimed.

The accompanying drawings, which are incorporated in and constitute part of this specification, are included to illustrate and provide a further understanding of the method and system of the invention. Together with the written description, the drawings serve to explain the principles of the invention.

### BRIEF DESCRIPTION OF THE DRAWINGS

FIG. 1 is an enlarged side view of a representative embodiment of the head portion of the fastener in accordance with the invention.

FIG. 2 is a side view of the complete fastener shown in FIG. 1.

FIG. 3 is an enlarged view of a perspective view of the fastener head portion shown in FIG. 1.

FIG. 4 is a perspective view of a representative embodiment of the collar (or tulip) in accordance with the invention.

FIG. 5 is a cross-sectional view of the collar shown in FIG. 4.

FIG. 6 is a side view of the collar shown in FIG. 4.

FIG. 7 is a bottom view of the collar shown in FIG. 4.

FIGS. 8A-8C depict schematic views of a method of positioning the fastener in the collar in accordance with the invention.

FIG. 9 is a perspective view of a representative embodiment of a fastener in accordance with the invention.

FIG. 10 is a front view of the fastener shown in FIG. 9.

FIG. 11 is a side view of the fastener shown in FIG. 9.

FIG. 12A is another front view of the fastener in accordance with the invention.

FIG. 12B is a perspective view taken along line B-B in FIG. 12A.

FIG. 12C is a cross-sectional view taken along line C-C in FIG. 12A.

FIG. 12D is a top view of the fastener shown in FIG. 12A.

FIG. 12E is a bottom view of the fastener shown in FIG. 12A.

FIG. 13A is another perspective view of a representative embodiment of the collar in accordance with the invention.

FIG. 13B is a front of the collar shown in FIG. 13A.

FIG. 13C is a cross-sectional view taken along line A-A in FIG. 13B.

FIG. 13D is a bottom view of the collar shown in FIG. 13A.

FIG. 13E is a side view of the collar shown in FIG. 13A.

FIG. 13F is a cross-sectional view taken along line B-B in FIG. 13E.

FIG. 13G is an enlarged view of section C in FIG. 13F.

### DETAILED DESCRIPTION OF THE PREFERRED EMBODIMENT

Reference will now be made in detail to the present preferred embodiments of the invention, an example of which is illustrated in the accompanying drawings. The method and corresponding steps of the invention will be described in conjunction with the detailed description of the system.

A spinal stabilization system can be installed in a patient to stabilize a portion of a spine. Spinal stabilization can be used, but is not limited to use, in patients having degenerative disc disease, spinal stenosis, spondylolisthesis, pseudoarthrosis, and/or spinal deformities; in patients having fracture or other vertebral trauma; and in patients after tumor resection. A spinal stabilization system can be installed using a minimally invasive procedure. An instrumentation set can include instruments and spinal stabilization system components for forming a spinal stabilization system in a patient.

A minimally invasive procedure can be used to limit an amount of trauma to soft tissue surrounding vertebrae that are to be stabilized. In some embodiments, the natural flexibility of skin and soft tissue can be used to limit the length and/or depth of an incision or incisions needed during the stabilization procedure. Minimally invasive procedures can provide limited direct visibility in vivo. Forming a spinal stabilization system using a minimally invasive procedure can include using tools to position system components in the body.

A minimally invasive procedure can be performed after installation of one or more spinal implants in a patient. The spinal implant or spinal implants can be inserted using an anterior procedure and/or a lateral procedure. The patient can be turned and a minimally invasive procedure can be used to install a posterior spinal stabilization system. A minimally invasive procedure for stabilizing the spine can be performed without prior insertion of one or more spinal implants in some patients. In some patients, a minimally invasive procedure can be used to install a spinal stabilization system after one or more spinal implants are inserted using a posterior spinal approach.

A spinal stabilization system can be used to achieve rigid pedicle fixation while minimizing the amount of damage to surrounding tissue. In some embodiments, a spinal stabilization system can be used to provide stability to two adjacent vertebrae (i.e., one vertebral level). A spinal stabilization system can include two bone fastener assemblies. One bone fastener assembly can be positioned in each of the vertebrae to be stabilized. An elongated member can be coupled and secured to the bone fastener assemblies. As used herein, “coupled” components can directly contact each other or can be separated by one or more intervening members. In some embodiments, a single spinal stabilization system can be installed in a patient. Such a system can be referred to as a unilateral, single-level stabilization system or a single-level, two-point stabilization system. In some embodiments, two spinal stabilization systems can be installed in a patient on opposite sides of a spine. Such a system can be referred to as a bilateral, single-level stabilization system or a single-level, four-point stabilization system.

In some embodiments, a spinal stabilization system can provide stability to three or more vertebrae (i.e., two or more vertebral levels). In a two vertebral level spinal stabilization system, the spinal stabilization system can include three bone fastener assemblies. One bone fastener assembly can be positioned in each of the vertebrae to be stabilized. An elongated member can be coupled and secured to the three bone fastener assemblies. In some embodiments, a single two-level spinal stabilization system can be installed in a patient. Such a system can be referred to as a unilateral, two-level stabilization system or a two-level, three-point stabilization system. In some embodiments, two three-point spinal stabilization systems can be installed in a patient on opposite sides of a spine. Such a system can be referred to as a bilateral, two-level stabilization system or a two-level, six-point stabilization system.

In some embodiments, combination systems can be installed. For example, a two-point stabilization system can be installed on one side of a spine, and a three-point stabilization system can be installed on the opposite side of the spine. The composite system can be referred to a five-point stabilization system.

Minimally invasive procedures can reduce trauma to soft tissue surrounding vertebrae that are to be stabilized. Only a small opening can need to be made in a patient. For example, for a single-level stabilization procedure on one side of the spine, the surgical procedure can be performed through a 2 cm to 4 cm incision formed in the skin of the patient. In some embodiments, the incision can be above and substantially between the vertebrae to be stabilized. In some embodiments, the incision can be above and between the vertebrae to be stabilized. In some embodiments, the incision can be above and substantially halfway between the vertebrae to be stabilized. Dilators, a targeting needle, and/or a tissue wedge can be used to provide access to the vertebrae to be stabilized without the need to form an incision with a scalpel through muscle and other tissue between the vertebrae to be stabilized. A minimally invasive procedure can reduce an amount of post-operative pain felt by a patient as compared to invasive spinal stabilization procedures. A minimally invasive procedure can reduce recovery time for the patient as compared to invasive spinal procedures.

Components of spinal stabilization systems can be made of materials including, but not limited to, titanium, titanium alloys, stainless steel, ceramics, and/or polymers. Some components of a spinal stabilization system can be autoclaved and/or chemically sterilized. Components that can not be autoclaved and/or chemically sterilized can be made of sterile materials. Components made of sterile materials can be placed in working relation to other sterile components during assembly of a spinal stabilization system.

Spinal stabilization systems can be used to correct problems in lumbar, thoracic, and/or cervical portions of a spine. Various embodiments of a spinal stabilization system can be used from the C1 vertebra to the sacrum. For example, a spinal stabilization system can be implanted posterior to the spine to maintain distraction between adjacent vertebral bodies in a lumbar portion of the spine.

In accordance with an aspect of the invention; a bone fastener assembly is provided. The bone fastener assembly includes a bone fastener, and a collar. The collar has an upper portion with a slot to receive an elongated member, a lower portion having a socket formed therein and an opening with a predetermined shape in communication with the socket, and a first longitudinal axis extending through the upper and lower portions. The bone fastener has a head portion to be received in the socket, and a shank portion to be attached to a vertebrae. The head portion has a first cross-sectional shape in a plane generally perpendicular to a second longitudinal axis extending through the head and shank portions of the fastener. The head portion further includes a second cross-sectional shape in a plane angled relative to the second longitudinal axis. The first cross-sectional shape is configured to prohibit movement of the head portion through the opening in the collar and the second cross-sectional shape is configured to allow movement of the head portion through the opening.

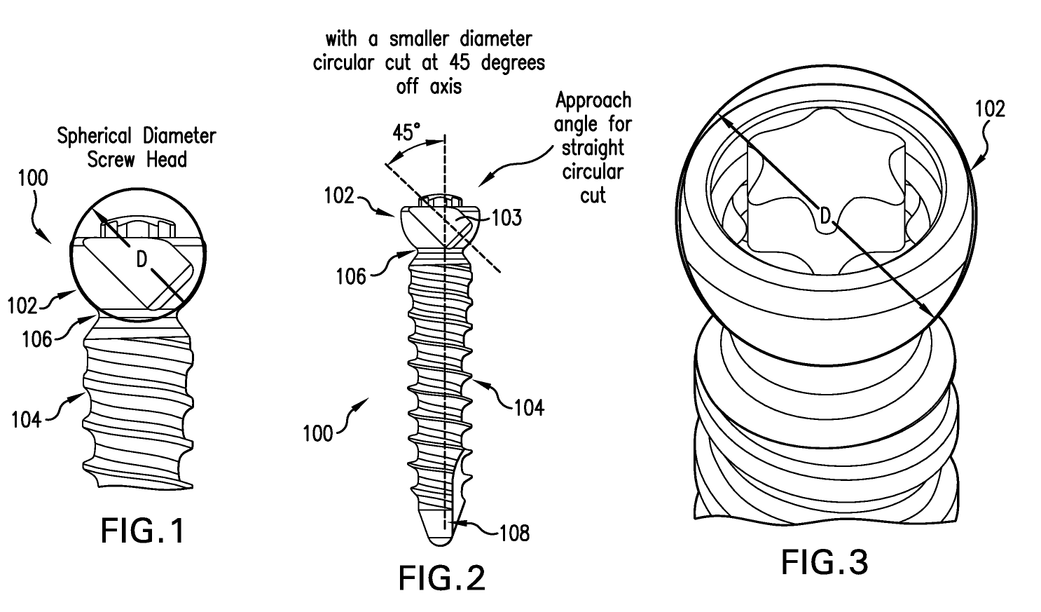

For purpose of explanation and illustration, and not limitation, an exemplary embodiment of the bone fastener assembly is shown in the accompanying figures. For example, FIGS. 1-3 illustrate a bone fastener in accordance with the invention shown generally by reference character 100. As shown in FIG. 2, the fastener 100 generally includes a head portion 102, a shank portion 104 and a neck portion 106 disposed therebetween. Alternative anchor members, such as hooks are contemplated to be within the scope of the present invention.

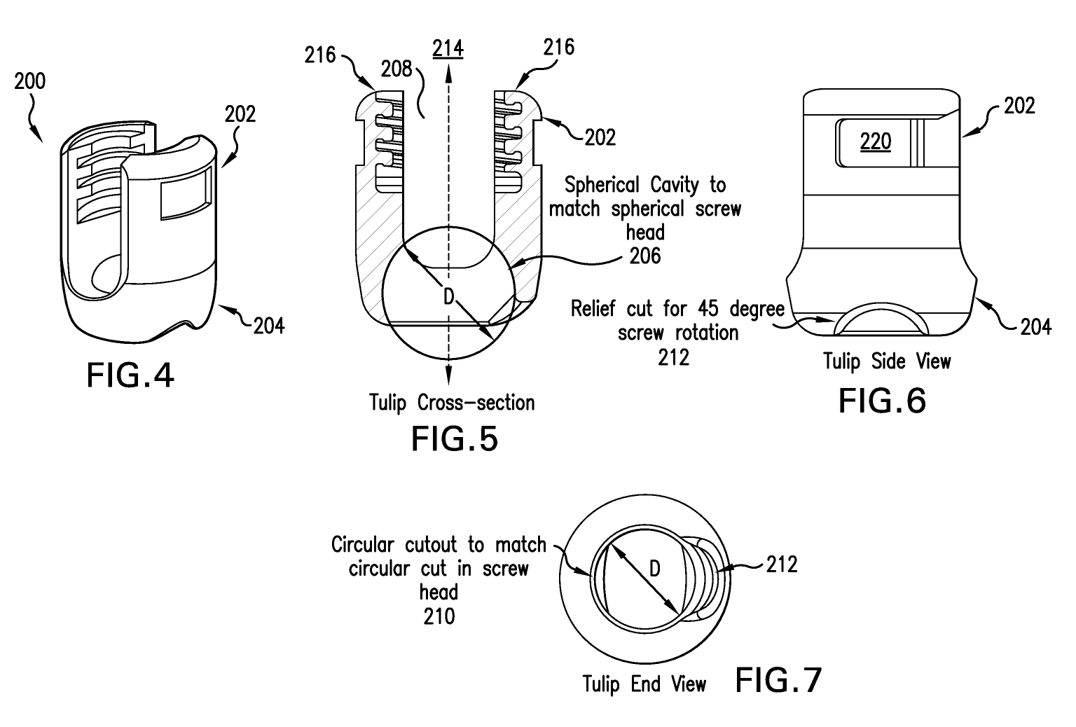

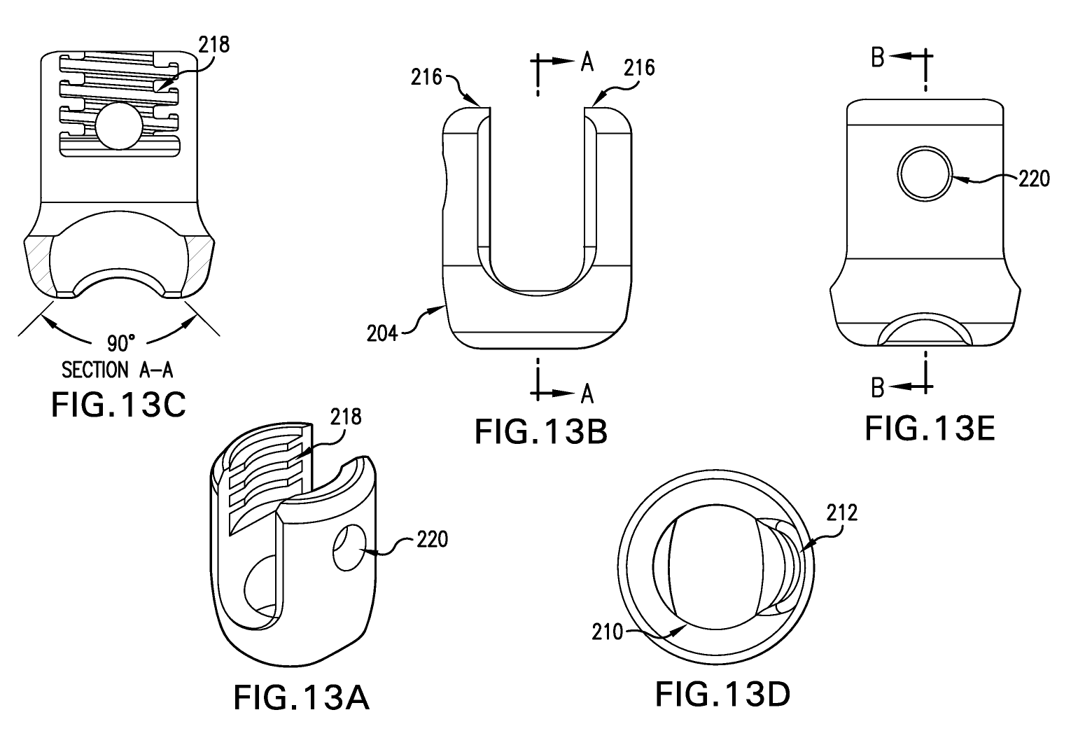

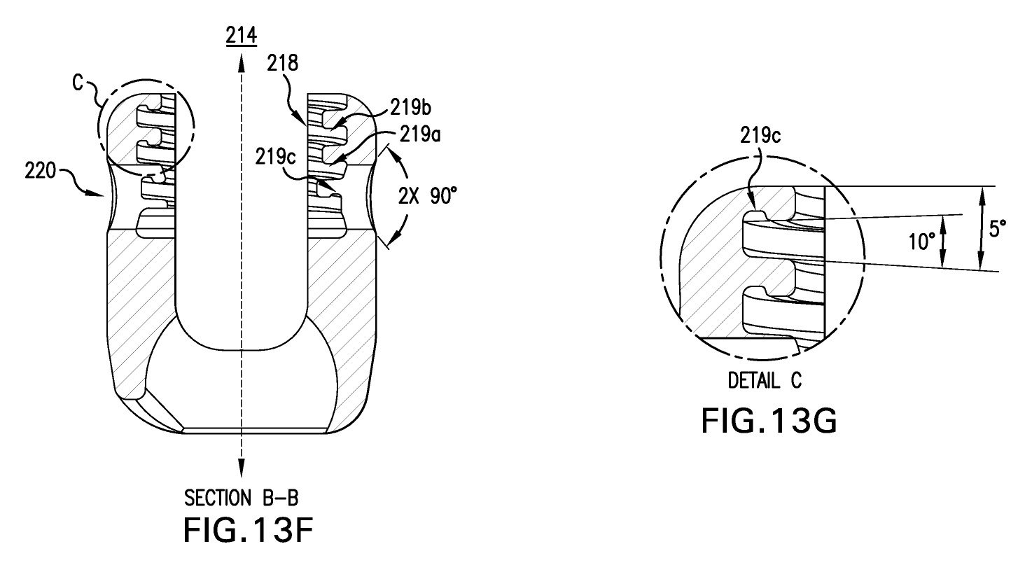

FIGS. 4-7 illustrate a collar in accordance with the invention shown generally by reference character 200, having an upper portion 202 with a slot 208 for receiving an elongated member (not shown), and a lower portion 204 having socket 206 formed therein for receiving the head of the fastener. The lower portion 204 includes an opening of predetermined shape such as a generally circular shape 210 and a relief 212 extending from the circular opening, as will be discussed in further detail below. A first longitudinal axis 214 extends between upper and lower portions and through the opening as shown in FIGS. 5 and 13F. Preferably, the opening 210 is oriented generally perpendicular to the first longitudinal axis 214. The relief extends upwardly and outwardly a distance into the lower portion 204 of the collar.

In accordance with a particular aspect of the present invention, the fastener has a second longitudinal axis 108, which extends through head portion 102 and shank portion 104 as embodied herein. Further, head portion 102 includes a first cross-sectional shape in a plane perpendicular to the second longitudinal axis 108, and a second cross-sectional shape in a plane angled relative to the second longitudinal axis.

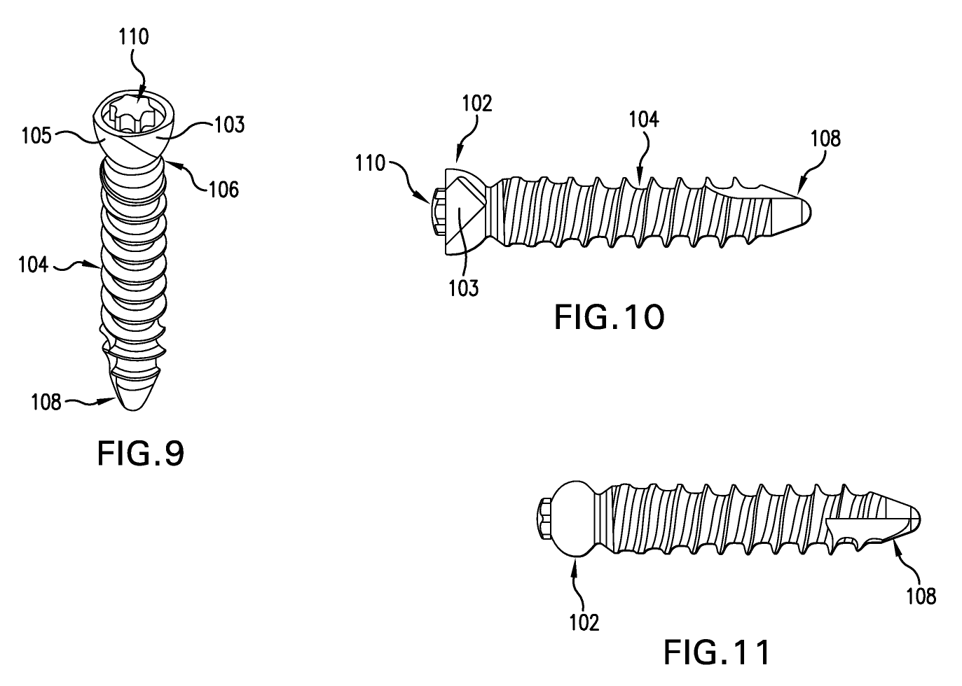

In a preferred embodiment, the second cross-sectional plane is angled 45° to the second longitudinal axis 108, as shown in FIG. 2. For example, the second cross-section is formed by machining a circular cut into the head 102 at a 45° angle to form facet 103, as illustrated in FIGS. 9-12D. Consequently, the diameter of the head 102 is smaller at the facet 103 than at spherical portion 105. Preferably, the second cross-sectional shape of the head, which coincides with facet 103, has a cross dimension which is approximately 90% of the diameter of the first cross-sectional shape. In one embodiment, the first cross-sectional shape relates to a generally spherical surface having a diameter of approximately 0.320 inches, and the second cross-sectional shape relates to a generally non-spherical surface, such as a cylindrical shape having a diameter of approximately 0.285 inches.

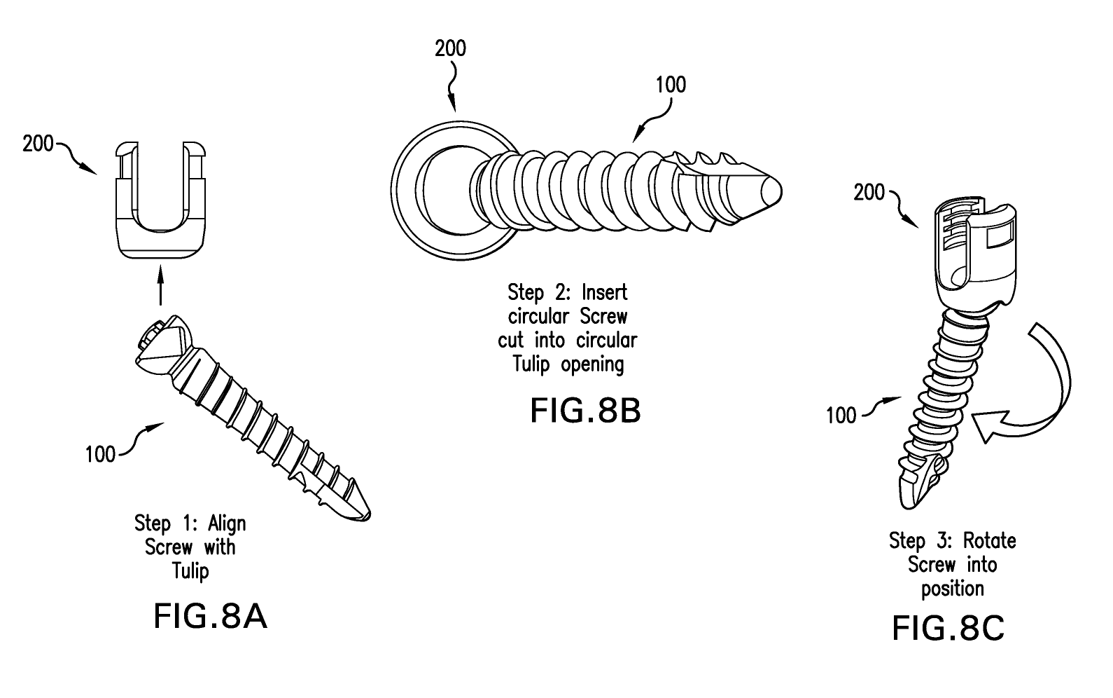

In accordance with another aspect of the invention, the opening in the bottom of the collar 200 is configured to permit insertion of the fastener 100 only when the circular cut is aligned with the opening at a particular angle, as illustrated in FIGS. 8A-C. In other words, when the circular cut, or facet 103, is aligned with the circular opening 210, the larger diameter portion 105 of head 102 registers with the relief cut extension 212 to permit insertion of the fastener from the bottom of the collar. Therefore, the larger diameter portion 105 can serve as a key to ensure proper alignment of the fastener and collar. In a preferred embodiment, the circular opening 210 in the bottom of the collar has a diameter of approximately 0.285 inches, which correlates to the diameter of the second cross-sectional shape formed by facet 103.

In accordance with another aspect of the invention, upon insertion of the fastener 100 into the collar such that the head 102 is received in the socket 206, the fastener can be rotated so that the second cross-sectional shape is no longer aligned with the opening. Accordingly, the fastener head 102 is received by the socket and prohibited from moving through the opening. For example, the fastener can be rotated 45° to ensure that the larger diameter portion 105 of the head or key, is displaced from the relief 212 and the smaller diameter facet is displaced from the circular portion of the opening. The fastener can be rotated relative to the second longitudinal axis extending through the fastener member. Alternatively, the fastener can be rotated relative to the plane of the opening in the collar.

Although reference is made to the opening having a circular portion and a relief and the second cross-sectional shape having a circular facet and key, alternative corresponding shapes are contemplated to be within the scope of the present invention.

A bone fastener can be, but is not limited to, a bone screw, a ring shank fastener, a barb, a nail, a brad, or a trocar. Bone fasteners and/or bone fastener assemblies can be provided in various lengths in an instrumentation set to accommodate variability in vertebral bodies. For example, an instrumentation set for stabilizing vertebrae in a lumbar region of the spine can include bone fastener assemblies with lengths ranging from about 30 mm to about 75 mm in 5 mm increments. A bone fastener assembly can be stamped with indicia (i.e., printing on a side of the collar). In some embodiments, a bone fastener assembly or a bone fastener can be color-coded to indicate a length of the bone fastener. In certain embodiments, a bone fastener with a 30 mm thread length can have a magenta color, a bone fastener with a 35 mm thread length can have an orange color, and a bone fastener with a 55 mm thread length can have a blue color. Other colors can be used as desired.

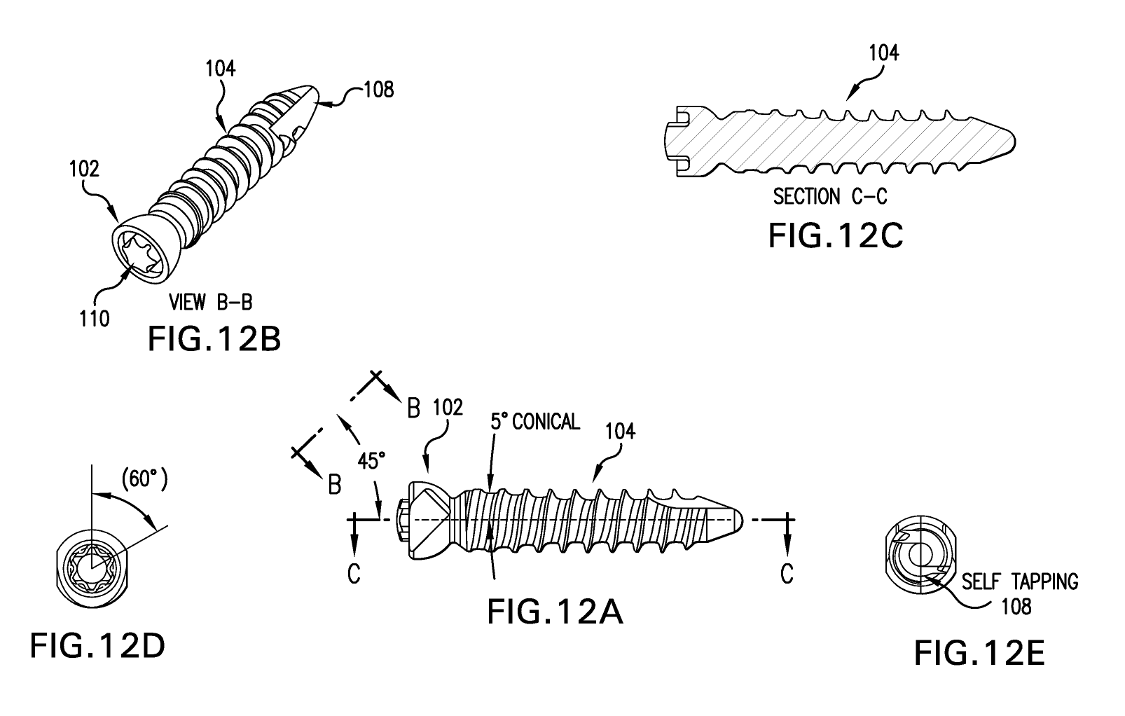

FIGS. 1-3, and 8-12E depict an embodiment of bone fastener 100 wherein the shank 104 includes thread. In some embodiments, the threads can include self-tapping start 108, as best shown in FIG. 12E. Self-tapping start 108 can facilitate insertion of bone fastener 108 into vertebral bone. Each bone fastener provided in an instrumentation set can have substantially the same thread profile and thread pitch. In an embodiment, the thread can have about a 4 mm major diameter and about a 2.5 mm minor diameter with a cancerous thread profile. In certain embodiments, the minor diameter of the thread can be in a range from about 1.5 mm to about 4 mm or larger. In certain embodiments, the major diameter of the thread can be in a range from about 3.5 mm to about 6.5 mm or larger. Bone fasteners with other thread dimensions and/or thread profiles can also be used. A thread profile of the bone fasteners can allow bone purchase to be maximized when the bone fastener is positioned in vertebral bone.

Head portion 102 of bone fastener 100 can include various configurations to engage a driver that inserts the bone fastener into a vertebra. In some embodiments, the driver can also be used to remove an installed bone fastener from a vertebra. In some embodiments, head 100 can include one or more tool portions 110, as shown in FIG. 9. Tool portions 110 can be recesses and/or protrusions designed to engage a portion of the driver. In some embodiments, bone fastener 100 can be cannulated for use in a minimally invasive procedure.

Neck 106 of bone fastener 100 can have a smaller diameter than adjacent portions of head 102 and shank 104. The diameter of neck 106 can fix the maximum angle that the collar of the bone fastener assembly can be rotated relative to bone fastener 100. In some embodiments, neck 106 can be sized to allow up to about 40° or more of angulation of the collar relative to the bone fastener. In some embodiments, the neck can be sized to allow up to about 30° of angulation of the collar relative to the bone fastener. In some embodiments, the neck can be sized to allow up to about 20° of angulation of the collar relative to the bone fastener.

The outer surface of the head 102 can have a smooth finish. In some embodiments, the outer surface can be surface treated, such as heavy grit blasting, or include coatings and/or coverings. Surface treatments, coatings, and/or coverings can be used to adjust frictional and/or wear properties of the outer surface of the head. In some embodiments, a portion of the outer surface of the head can be shaped and/or textured to limit a range of motion of the fastener relative to a collar of a bone fastener assembly.

As discussed above, spinal stabilization systems can include bone fastener assemblies having bone fasteners 100, collars 200, elongated member (not shown), and/or closure members (not shown). Other spinal stabilization system embodiments can include, but are not limited to, plates, dumbbell-shaped members, and/or transverse connectors.

As used herein, the term “collar” includes any element that wholly or partially encloses or receives one or more other elements. A collar can enclose or receive elements including, but not limited to, a bone fastener, a closure member, and/or an elongated member. A collar can have any of various physical forms. In some embodiments, a collar can have a “U” shape, however it is to be understood that a collar can also have other shapes. A collar can be open or closed. A collar having a slot and an open top, such as collar 200 shown in FIGS. 4-7 and 13A-F, can be referred to as an “open collar”. A bone fastener assembly that includes an open collar can be referred to as an “open fastener”. In some embodiments, an elongated member (not shown) can be top loaded into the open fastener. A closure member (not shown) can be coupled to the collar to secure the elongated member to the open fastener.

Alternatively, a collar that does not include a slot and an open top can be referred to as a “closed collar”. A spinal implant that includes a closed collar can be referred to as a “closed implant”. A closed collar can include an aperture, bore, or other feature in side surfaces for accommodating other components of a stabilization system (e.g., an elongated member). A set screw can be used to securely couple an elongated member to a closed implant.

In a preferred embodiment of the invention, collar 200 includes a body portion and arms 216. Arms 216 can extend from the lower body portion 204 as shown in FIGS. 4-5 and 13F. Body portion 204 of collar 200 can be greater in width than a width across arms 216 of collar 200 (i.e., body 204 can have a maximum effective outer diameter greater than a maximum effective outer diameter of arms 216). A reduced width across arms 216 allows a detachable member to be coupled to the arms without substantially increasing a maximum effective outer diameter along a length of collar 200. Thus, a reduced width across arms 216 can reduce bulk at a surgical site.

A height of body 204 can range from about 3 millimeters (mm) to about 7 mm. In an embodiment, a height of body 204 is about 5 mm. In a preferred embodiment the inner surface of collar 200 which defines the socket 206 can be machined to complement a portion of an outer surface of the fastener 100 that is to be positioned in collar 200. Machining of socket 206 can enhance retention of the fastener in the collar 200. Additionally, socket 206 can be complementary in shape to a portion of outer surface of fastener head 102 (see FIG. 5) so that the fastener is able to swivel in the collar. Inner surfaces and/or outer surfaces of collar 200 can be surface treated or include coatings and/or coverings to modify frictional properties or other properties of the collar.

A portion of the collar, and preferably the arms, can include a thread to receive a corresponding closure member. In a preferred embodiment, inner surfaces of arms 216 can include modified thread 218. Modified threads 218 can engage complementary modified threads of a closure member (not shown) to secure an elongated member (not shown) to a bone fastener assembly. Modified threads 218 can have a constant pitch or a variable pitch, as disclosed in U.S. Patent Application Publication No. 2006/0084993.

A height and a width of arms 216 can be sized as needed, for example, arms 216 can range in height from about 8 mm to about 15 mm. In an embodiment, a height of arms 216 is about 11 mm. A width (i.e., effective diameter) of arms 216 can range from about 5 mm to 14 mm. Arms 216 and body 204 form slot 208 which can be sized to receive an elongated member. Slot 208 can include, but is not limited to, an elongated opening of constant width, an elongated opening of variable width, a rectangular opening, a trapezoidal opening, a circular opening, a square opening, an ovoid opening, an egg-shaped opening, a tapered opening, and combinations and/or portions thereof. In some embodiments, a first portion of slot 208 can have different dimensions than a second portion of slot 208. In certain embodiments, a portion of slot 208 in first arm 216 can have different dimensions than a portion of slot 208 in second arm 216. When an elongated member is positioned in slot 208, a portion of the elongated member can contact a head of a bone fastener positioned in the collar.

In an embodiment of a collar, arms 216 of collar 200 can include one or more openings and/or indentions 220, as shown in FIGS. 6 and 13A. Indentions 220 can vary in size and shape (e.g., circular, triangular, rectangular). Indentions 220 can be position markers and/or force application regions for instruments that perform reduction, compression, or distraction of adjacent vertebrae. In some embodiments, openings and/or indentions can be positioned in the body of the collar.

In accordance with a preferred embodiment, the bone fastener is rotatably positioned in a collar such that the bone fastener is able to move radially and/or rotationally relative to the collar (or the collar relative to the bone fastener) within a defined range of motion. The range of motion can be provided within a plane, such as by a hinged connection, or within a three-dimensional region, such as by a ball and socket connection. Motion of the bone fastener relative to the collar (or the collar relative to the bone fastener) is referred to as “angulation” and/or “polyaxial movement”.

Preferably, a closure member (not shown) is coupled to a collar of a bone fastener assembly to fix an elongated member (not shown) positioned in the collar to the bone fastener assembly. In some embodiments, a closure member can be cannulated. In certain embodiments, a closure member can have a solid central core. A closure member with a solid central core can allow more contact area between the closure member and a driver used to couple the closure member to the collar. A closure member with a solid central core can provide a more secure connection to an elongated member than a cannulated closure member by providing contact against the elongated member at a central portion of the closure member as well as near an edge of the closure member.

A bottom surface of a closure member preferably includes structure and/or texturing that promotes contact between the closure member and an elongated member. A portion of the structure and/or texturing can enter and/or deform an elongated member when the closure member is coupled to the elongated member. Having a portion of the closure member enter and/or deform the elongated member can couple the elongated member to the closure member and a bone fastener assembly so that movement of the elongated member relative to the bone fastener assembly is inhibited.

The closure member can couple to collar 200 by a variety of systems including, but not limited to, standard threads, modified threads, reverse angle threads, buttress threads, or helical flanges. A buttress thread on a closure member can include a rearward-facing surface that is substantially perpendicular to the axis of the closure member. Additionally, closure member can be advanced into an opening in a collar to engage a portion of elongated member. In some embodiments, the closure member can inhibit movement of elongated member relative to collar 200.

FIGS. 5 and 13F-G depict a cross-sectional view of collar 200 having a female modified thread configured to receive a male modified thread (not shown) of a closure member. Male modified thread includes male distal surface and male proximal surface. Collar 200 includes female modified thread 218 on an inside surface of arms 216. Female modified thread 218 includes female proximal surface 219a and female distal surface 219b. Male proximal surface can couple to female distal surface 219a during use. Male proximal surface and female distal surface 219b can be load-bearing surfaces. A load can result from an upward load on the closure member, such as a load resulting when the elongated member positioned in a slot 208 of collar 200 is secured to the bone fastener assembly by the closure member.

Raised portions can be included on the male distal surface, and corresponding recessed portions 219c can be included on female proximal surface 219a. Cooperating surfaces of modified threads 218 can contact or be proximate to one another during use. As used herein, “proximate” means near to or closer to one portion of a component than another portion of a component. Engagement of cooperating surfaces of modified threads 218 during use can inhibit radial expansion of collar 200. Engagement of cooperating surfaces can inhibit spreading of arms 216 away from each other (i.e., inhibit separation of the arms). In some embodiments, cooperating surfaces can be substantially parallel to a central axis of the closure member. In other embodiments, cooperating surfaces can be angled relative to a central axis of the closure member.

In an embodiment, a bone fastener assembly and a closure member can be coupled with a running fit. A running fit (i.e., a fit in which parts are free to rotate) can result in predictable loading characteristics of a coupling of a bone fastener assembly and a closure member. Predictable loading characteristics can facilitate use of a closure member with a break-off portion designed to shear off at a predetermined torque. A running fit can also facilitate removal and replacement of closure members. In some embodiments, a closure member can include an interference fit (e.g., crest-to-root radial interference).

Various instruments can be used in a minimally invasive procedure to form a spinal stabilization system in a patient. Further description of these tools and the accompanying methods for performing the minimally invasive procedure are disclosed in U.S. patent application Ser. Nos. 10/697,793 filed Oct. 30, 2003; 11/284,282 filed Nov. 21, 2005; and 11/337,863 filed Jan. 23, 2006; the entire disclosures of each are hereby incorporated by reference.

It will be apparent to those skilled in the art that various modifications and variations can be made in the method and system of the present invention without departing from the spirit or scope of the invention. Thus, it is intended that the present invention include modifications and variations that are within the scope of the appended claims and their equivalents.

1. A bone fastener assembly comprising:
a collar having an upper portion with a slot formed therein to receive an elongated member, a lower portion having a socket formed therein and an opening with a predetermined shape in communication with the socket, and a first longitudinal axis extending through the upper and lower portions;
a fastener member having a head portion to be received in the socket, a shank portion to be attached to a vertebrae, and a second longitudinal axis extending through the head portion and shank portion of the fastener member, the head portion of the fastener member having a first cross-sectional shape in a plane generally perpendicular to the second longitudinal axis and a second cross-sectional shape in a plane angled relative to the second longitudinal axis,
the first cross-sectional shape configured to prohibit movement of the head portion through the opening and the second cross-sectional shape configured to allow movement of the head portion through the opening.

2. The bone fastener assembly of claim 1, wherein the head portion mates with the socket for polyaxial movement of the fastener member relative to the collar.

3. The bone fastener assembly of claim 1, wherein the opening is located at a bottom surface of the collar; the fastener member being received in the socket through the opening.

4. The bone fastener assembly of claim 1, wherein the second cross sectional shape of the head of the fastener member is in a plane angled about 45° relative to the second longitudinal axis.

5. The bone fastener assembly of claim 1, wherein the second cross-sectional shape of the head portion has a diameter of approximately 90% of the diameter of the first cross-sectional shape of the head portion.

6. The bone fastener assembly of claim 1, wherein at least a portion of the first cross-sectional shape of the head portion has a diameter of approximately 0.320 inches.

7. The bone fastener assembly of claim 1, wherein at least a portion of the second cross-sectional shape of the head portion has a diameter of approximately 0.285 inches.

8. The bone fastener assembly of claim 1, wherein the predetermined shape of the opening includes a first portion having a generally circular shape in a plane perpendicular to the first longitudinal axis, and a second portion having a relief extending from the generally circular shape.

9. The bone fastener assembly of claim 8, wherein the second cross-sectional shape of the fastener member is keyed to the generally circular shape of the collar.

10. The bone fastener assembly of claim 8, wherein the generally circular opening has a diameter of approximately 0.285 inches.

11. The bone fastener assembly of claim 1, wherein the upper portion of the collar includes a plurality of arms, each arm including a threaded portion.

12. The bone fastener assembly of claim 11, further comprising a closure member having a thread to engage the threaded portion of the collar.

13. The bone fastener assembly of claim 12, wherein the thread of the collar includes a female modified thread including a female proximal surface and a female distal surface, and the closure member includes a male modified thread including a male proximal surface and a male distal surface, and wherein the male proximal surface of the closure member is configured to couple with the female distal surface of the collar, the female proximal surface and the male distal surface each comprise at least one raised portion, and wherein one or more surfaces of such raised portions are configured to couple during use to inhibit radial expansion of the collar.

14. A method for assembling a bone fastener system comprising:
providing a collar having an upper portion with a slot formed therein to receive an elongated member, a lower portion having a socket formed therein and an opening with a predetermined shape in communication with the socket, and a first longitudinal axis extending through the upper and lower portions;
providing a fastener member having a head portion to be received in the socket, a shank portion to be attached to a vertebrae, and a second longitudinal axis extending through the head portion and shank portion of the fastener member, the head portion of the fastener member having a first cross-sectional shape in a plane generally perpendicular to the second longitudinal axis and a second cross-sectional shape in a plane angled relative to the second longitudinal axis;
aligning the collar with the fastener member with the second cross-sectional shape of the head portion coinciding with the opening;
inserting the head portion of the fastener member into the socket through the opening in the lower portion of the collar;
rotating the fastener to misalign the second cross-sectional shape of the head portion from the opening of the collar to prohibit movement of the head portion through the opening.

15. The method of claim 14, wherein the first longitudinal axis extends through the opening, and the head portion is aligned with the plane of the second cross-sectional shape perpendicular to the first longitudinal axis.

16. The method of claim 14, wherein the predetermined shape of the collar opening is configured to allow for insertion of the head portion of the fastener member, and inhibit removal of the head portion of the fastener from the collar upon approximately 45° rotation of the fastener member.

17. The method of claim 14, wherein the fastener member is rotated relative to the second longitudinal axis,

18. The method of claim 14, wherein the fastener member is rotated relative to the first longitudinal axis.

19. The method of claim 14, wherein the predetermined shape of the collar opening includes a first portion having a generally circular shape in a plane perpendicular to the first longitudinal axis, and a second portion having a relief extending from the generally circular shape.

20. The method of claim 19, wherein the head portion is configured with the second cross-sectional shape keyed to the generally circular opening, the head portion of the fastener member aligned with the opening, inserted into the socket, and rotated.

21. A spinal fixation system comprising:
a collar having an upper portion with a slot formed therein to receive an elongated member, a lower portion having a socket formed therein and an opening with a predetermined shape in communication with the socket, and a first longitudinal axis extending through the upper and lower portions;
a fastener member having a head portion to be received in the socket, a shank portion to be attached to a vertebrae, and a second longitudinal axis extending through the head portion and shank portion of the fastener member, the head portion of the fastener member having a first cross-sectional shape in a plane generally perpendicular to the second longitudinal axis and a second cross-sectional shape in a plane angled relative to the second longitudinal axis,
the first cross-sectional shape configured to prohibit movement of the head portion through the opening and the second cross-sectional shape configured to allow movement of the head portion through the opening;
an elongated rod to be disposed within the slot;
a closure member having a thread to engage a threaded portion of the collar and secure the elongated member within the slot of the collar.
